(19) United States
(12) Patent Application Publication
(10) Pub. No.: US 2011/0270006 A1
(43) Pub. Date: Nov. 3, 2011

(54) Use of an Additive in the Coupling of Toluene with a Carbon Source

(75) Inventors: Joseph E. Pelati, Houston, TX (US); Sivadinarayana Chinta, Missouri City, TX (US)

(73) Assignee: FINA TECHNOLOGY, INC., Houston, TX (US)

(21) Appl. No.: 13/113,064

(22) Filed: May 22, 2011

Related U.S. Application Data
(63) Continuation-in-part of application No. 12/938,449, filed on Nov. 3, 2010.
Application No. 12/938,449 is a continuation-in-part of application No. 12/763,234, filed on Apr. 20, 2010.

(51) Int. Cl. C07C 2/00; B01J 29/04; B01J 23/78; B01J 23/26; C01B 39/00; B01J 23/28; B01J 23/14; B01J 23/18; B01J 23/66; B01J 37/30; B01J 23/04; B01J 23/10

(52) U.S. Cl. 585/438; 502/344; 502/87; 502/330; 502/317; 502/304; 502/347; 502/345; 502/11; 502/74; 502/73; 423/700

(57) ABSTRACT

A method is disclosed of preparing a catalyst, including contacting a substrate with at least one solution including a first promoter being Cs and at least one solution including a second promoter. The contact subjects the substrate to the addition of the first and second promoters, thereby forming the catalyst comprising the first and second promoters. In the method disclosed, the second promoter is capable of undergoing a redox reaction.

### CROSS-REFERENCE TO RELATED APPLICATIONS

The present invention is a continuation-in-part (CIP) of commonly-owned co-pending application U.S. patent application Ser. No. 12/938,449 filed by Fina Technology, Inc. on Nov. 3, 2010, which in turn is a continuation-in-part (CIP) of commonly-owned co-pending U.S. patent application Ser. No. 12/763,234 filed by Fina Technology, Inc. on Apr. 20, 2010.

### FIELD

The present invention relates to a method for the production of styrene and ethylbenzene. More specifically, the invention relates to the alkylation of toluene with a carbon source (herein referred to as a C₁ source) such as methanol and/or formaldehyde, to produce styrene and ethylbenzene.

### BACKGROUND

Styrene is a monomer used in the manufacture of many plastics. Styrene is commonly produced by making ethylbenzene, which is then dehydrogenated to produce styrene. Ethylbenzene is typically formed by one or more aromatic conversion processes involving the alkylation of benzene.

Aromatic conversion processes, which are typically carried out utilizing a molecular sieve type catalyst, are well known in the chemical processing industry. Such aromatic conversion processes include the alkylation of aromatic compounds such as benzene with ethylene to produce alkyl aromatics such as ethylbenzene. Typically an alkylation reactor, which can produce a mixture of monoalkyl and polyalkyl benzenes, will be coupled with a transalkylation reactor for the conversion of polyalkyl benzenes to monoalkyl benzenes. The transalkylation process is operated under conditions to cause disproportionation of the polyalkylated aromatic fraction, which can produce a product having an enhanced ethylbenzene content and reduced polyalkylated content. When both alkylation and transalkylation processes are used, two separate reactors, each with its own catalyst, can be employed for each of the processes.

Ethylene is obtained predominantly from the thermal cracking of hydrocarbons, such as ethane, propane, butane, or naphtha. Ethylene can also be produced and recovered from various refinery processes. Thermal cracking and separation technologies for the production of relatively pure ethylene can account for a significant portion of the total ethylbenzene production costs.

Benzene can be obtained from the hydrodealkylation of toluene that involves heating a mixture of toluene with excess hydrogen to elevated temperatures (for example 500° C. to 600° C.) in the presence of a catalyst. Under these conditions, toluene can undergo dealkylation according to the chemical equation: C₆H₅CH₃+H₂→C₆H₆+CH₄. This reaction requires energy input and as can be seen from the above equation, produces methane as a byproduct, which is typically separated and may be used as heating fuel for the process.

Another known process includes the alkylation of toluene to produce styrene and ethylbenzene. In this alkylation process, various aluminosilicate catalysts are utilized to react methanol and toluene to produce styrene and ethylbenzene. However, such processes have been characterized by having very low yields in addition to having very low selectivity to styrene and ethylbenzene.

Also, the aluminosilicate catalysts are typically prepared using solutions of acetone and other highly flammable organic substances, which can be hazardous and require additional drying steps. For instance a typical aluminosilicate catalyst can include various promoters supported on a zeolitic substrate. These catalysts can be prepared by subjecting the zeolite to an ion-exchange in an aqueous solution followed by a promoter metal impregnation using acetone. This method requires an intermediate drying step after the ion-exchange to remove all water prior to the promoter metal impregnation with acetone. After the promoter metal impregnation the catalyst is subjected to a further drying step to remove all acetone. This intermediate drying step typically involves heating to at least 150° C., which results in increased costs.

Additionally, the reaction of toluene with methanol includes a multi-step mechanism, wherein methanol is first converted to formaldehyde and hydrogen gas. The conversion of methanol to formaldehyde and hydrogen gas is a reduction/oxidation reaction sequence, hereinafter referred to as a “redox” reaction, which involves the loss of two electrons from the carbon moiety and the consumption of those two electrons by two protons (H⁺) to form hydrogen gas. Furthermore, the reaction of toluene and methanol can create an excess of hydroxide groups (—OH) on the surface if the formation of hydrogen gas lags the formation of formaldehyde and other such reactions. Such a buildup of hydroxide groups can lead to deactivation and by-product formation.

In view of the above, it would be desirable to have a process of producing styrene and/or ethylbenzene that does not rely on thermal crackers and expensive separation technologies as a source of ethylene. It would further be desirable to avoid the process of converting toluene to benzene with its inherent expense and loss of a carbon atom to form methane. It would be desirable to produce styrene without the use of benzene and ethylene as feedstreams. It would also be desirable to produce styrene and/or ethylbenzene in one reactor without the need for separate reactors requiring additional separation steps. Furthermore, it is desirable to achieve a process having a high yield and selectivity to styrene and ethylbenzene. Even further, it is desirable to achieve a process having a high yield and selectivity to styrene such that the step of dehydrogenation of ethylbenzene to produce styrene can be reduced. It is further desirable to be able to produce a catalyst having the properties desired without involving flammable materials and/or intermediate drying steps. Still yet, it would be desirable to achieve a process having a high yield and selectivity to styrene by producing a catalyst capable of providing a redox reaction, wherein the redox center of the catalyst can assist in the conversion of excess hydroxide groups to hydrogen gas to assist in preventing deactivation and by-product formation.

### SUMMARY

The present invention in its many embodiments relates to a process of making styrene. In an embodiment of the present invention, a method is provided for preparing a catalyst including contacting a substrate with at least one solution including a first promoter being Cs and at least one solution including a second promoter. The contact subjects the substrate to the addition of the first and second promoters, thereby forming the catalyst including the first and second promoters. The second promoter is capable of facilitating a redox reaction. The substrate can be a zeolite. Optionally, the catalyst is capable of effecting a reaction of at least a portion of a C₁ source with toluene to form a product stream including one or more of styrene or ethylbenzene.

In an embodiment, either by itself or in combination with any other embodiment, the method further includes a first solution including the first promoter being Cs and a second solution including the second promoter. The first solution and second solution can simultaneously contact the substrate resulting in simultaneous addition of the first and second promoters to the substrate, resulting in a substrate including the first and second promoters. Optionally, the method includes a first solution including the first promoter being Cs and a second solution including the second promoter. The first solution can initially contact the substrate resulting in the addition of the first promoter resulting in a substrate including the first promoter, followed by contacting the substrate including the first promoter with the second solution including the second promoter, resulting in ion exchange between the cationic sites on the substrate and the second promoter, finally resulting in a substrate including the first and second promoters.

In an embodiment, either by itself or in combination with any other embodiment, the second promoter is selected from the group of Fe, Cr, Ce, Mo, Sn, Bi, Ag, Cu, and combinations thereof. Optionally, the second promoter is selected from the group of Fe, Cu, and combinations thereof. The catalyst can include Cs in amounts ranging from 0.1 wt % to 33 wt % based on the total weight of the catalyst. Optionally, the substrate includes a third promoter selected from the group of Co, Mn, Ti, Zr, V, Nb, K, Ga, B, P, Rb, Na, Mg, and any combinations thereof

Another embodiment of the present invention includes a catalyst including a zeolitic support and a plurality of promoters including a first promoter being Cs and a second promoter selected from the group of Fe, Cr, Ce, Mo, Sn, Bi, Ag, Cu, and combinations thereof, wherein the second promoter is capable of facilitating a redox reaction. The plurality of promoters can be added onto the zeolitic support by ion exchange.

In an embodiment, either by itself or in combination with any other embodiment, the catalyst is capable of effecting a reaction of at least a portion of a C₁ source with toluene to form a product stream including one or more of styrene or ethylbenzene.

In an embodiment, either by itself or in combination with any other embodiment, the catalyst is capable of effecting selectivity to styrene of greater than 30 mol %.

In an embodiment, either by itself or in combination with any other embodiment, the second promoter can be selected from the group of Fe, Cu, and combinations thereof. The cesium can be present in the catalyst in amounts of from 0.1 to 33 wt % based on the total weight of the catalyst. The ion exchange can be performed in an aqueous medium utilizing water soluble promoter precursors.

In yet another embodiment of the present invention, a process for making styrene is provided including reacting toluene with the C₁ source in the presence of the catalyst in a reactor to form a product stream including ethylbenzene and styrene. The catalyst includes a first promoter being Cs and a second promoter supported on a zeolite, and the C₁ source is selected from the group of methanol, formaldehyde, formalin, trioxane, methylformcel, paraformaldehyde, methylal, dimethyl ether, and combinations thereof. The second promoter can be selected from the group of Fe, Cr, Ce, Mo, Sn, Bi, Ag, Cu, and combinations thereof. Optionally, the promoter is selected from the group of Fe, Cu, and combinations thereof.

The various aspects of the present invention can be joined in combination with other aspects of the invention and the listed embodiments herein are not meant to limit the invention. All combinations of aspects of the invention are enabled, even if not given in a particular example herein.

### BRIEF DESCRIPTION OF DRAWINGS

FIG. 1 illustrates a flow chart for the production of styrene by the reaction of formaldehyde and toluene, wherein the formaldehyde is first produced in a separate reactor by either the dehydrogenation or oxidation of methanol and is then reacted with toluene to produce styrene.

FIG. 2 illustrates a flow chart for the production of styrene by the reaction of formaldehyde and toluene, wherein methanol and toluene are fed into a reactor, wherein the methanol is converted to formaldehyde and the formaldehyde is reacted with toluene to produce styrene.

### DETAILED DESCRIPTION

In an aspect of the current invention, toluene is reacted with a carbon source capable of coupling with toluene to form ethylbenzene or styrene, which can be referred to as a C₁ source, to produce styrene and ethylbenzene. In an embodiment, the C₁ source includes methanol or formaldehyde or a mixture of the two. In an alternative embodiment, toluene is reacted with one or more of the following: formalin, trioxane, methylformcel, paraformaldehyde and methylal. In a further embodiment, the C₁ source is selected from the group of methanol, formaldehyde, formalin (37-50% H₂CO in solution of water and MeOH), trioxane (1,3,5-trioxane), methylformcel (55% H₂CO in methanol), paraformaldehyde and methylal (dimethoxymethane), dimethyl ether, and combinations thereof.

Formaldehyde can be produced either by the oxidation or dehydrogenation of methanol.

In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. The dehydrogenation process is described in the equation below:

CH₃OH→CH₂O+H₂

Formaldehyde can also be produced by the oxidation of methanol to produce formaldehyde and water. The oxidation of methanol is described in the equation below:

2CH₃OH+O₂→2CH₂O+2H₂O

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogenation of the formaldehyde back to methanol. Purified formaldehyde could then be sent to a styrene reactor and the unreacted methanol could be recycled.

Although the reaction has a 1:1 molar ratio of toluene and the C₁ source, the ratio of the feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range from 50:1 to 1:50; from 20:1 to 1:20; from 10:1 to 1:10; from 5:1 to 1:5; from 2:1 to 1:2. In a specific aspect, the ratio of toluene:C₁ source can range from 2:1 to 5:1.

The reaction of toluene with methanol to styrene has a multi-step mechanism carried out in a single reactor in an embodiment of the present invention. The methanol first is converted to formaldehyde and hydrogen gas. This is a reduction/oxidation (redox) reaction sequence that involves the loss of two electrons from the carbon moiety and the consumption of those two electrons by two protons (H+) to form hydrogen gas. Suitable catalysts for the toluene and methanol to styrene reaction are cesium-modified zeolites. The cesium is the chemically active site while the zeolite provides the shape selectivity for the catalyst. The Cs—O active site fundamentally is not a redox center so if a promoter is added that is redox active and can promote the transfer of electrons, then the formation of styrene from toluene and methanol will be enhanced. Such promoter species include but are not limited to Fe, Cu, Ag, V, Cr, Mn, Mo, Sn, Ga, Nb, Bi and Ce.

Also with time, the reaction can build up an excess of hydroxide groups (—OH) on the surface if the formation of hydrogen gas lags the formation of formaldehyde and other such reactions. The redox promoters can also help convert the excess hydroxide sites to hydrogen gas to help prevent deactivation and by-product formation. The reaction of toluene with methanol to styrene provided through the multi-step mechanism is as follows:

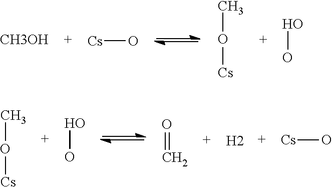

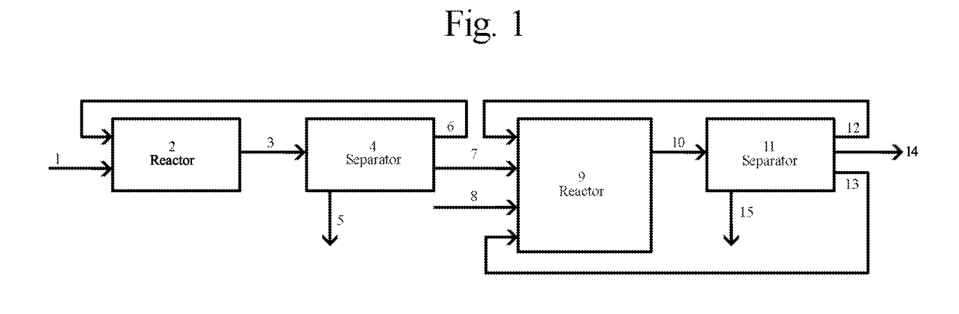

In FIG. 1 there is a simplified flow chart of one embodiment of the styrene production process described above. In this embodiment, a first reactor (2) is either a dehydrogenation reactor or an oxidation reactor. This reactor is designed to convert the first methanol feed (1) into formaldehyde. The gas product (3) of the reactor is then sent to a gas separation unit (4) where the formaldehyde is separated from any unreacted methanol and unwanted byproducts. Any unreacted methanol (6) can then be recycled back into the first reactor (2). The byproducts (5) are separated from the clean formaldehyde (7).

In one embodiment the first reactor (2) is a dehydrogenation reactor that produces formaldehyde and hydrogen and the separation unit (4) is a membrane capable of removing hydrogen from the product stream (3).

In an alternate embodiment the first reactor (2) is an oxidative reactor that produces product stream (3) comprising formaldehyde and water. The product stream (3) comprising formaldehyde and water can then be sent to the second reactor (9) without a separation unit (4).

The formaldehyde feed stream (7) is then reacted with a feed stream of toluene (8) in a second reactor (9). The toluene and formaldehyde react to produce styrene. The product (10) of the second reactor (9) may then be sent to an optional separation unit (11) where any unwanted byproducts (15) such as water can separated from the styrene, unreacted formaldehyde and unreacted toluene. Any unreacted formaldehyde (12) and the unreacted toluene (13) can be recycled back into the reactor (9). A styrene product stream (14) can be removed from the separation unit (11) and subjected to further treatment or processing if desired.

The operating conditions of the reactors and separators will be system specific and can vary depending on the feedstream composition and the composition of the product streams. The reactor (9) for the reaction of toluene and formaldehyde will operate at elevated temperatures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

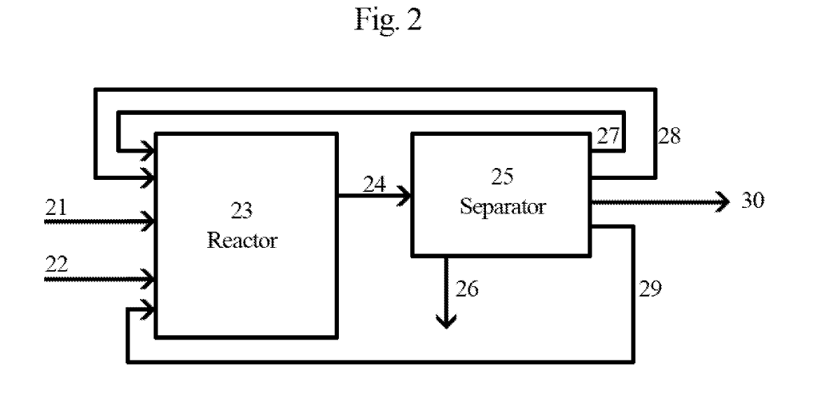

FIG. 2 is a simplified flow chart of another embodiment of the styrene process discussed above. A C₁ source containing feed stream (21) is fed along with a feed stream of toluene (22) in a reactor (23). Toluene and the C₁ source then react to produce styrene. The product (24) of the reactor (23) may then be sent to an optional separation unit (25) where any unwanted byproducts (26) can be separated from the styrene, and any unreacted C1 source, unreacted methanol, unreacted formaldehyde and unreacted toluene. Any unreacted methanol (27), unreacted formaldehyde (28) and the unreacted toluene (29) can be recycled back into the reactor (23). A styrene product stream (30) can be removed from the separation unit (25) and subjected to further treatment or processing if desired.

The operating conditions of the reactors and separators will be system specific and can vary depending on the feedstream composition and the composition of the product streams. The reactor (23) for the reactions of methanol to formaldehyde and toluene with a C₁ source, such as formaldehyde, will operate at elevated temperatures and and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

Improvement in side chain alkylation selectivity may be achieved by treating a molecular sieve zeolite catalyst with chemical compounds to inhibit the external acidic sites and minimize aromatic alkylation on the ring positions. Another means of improvement of side chain alkylation selectivity can be to inhibit overly basic sites. Another means of improvement of side chain alkylation selectivity can be to impose restrictions on the catalyst structure to facilitate side chain alkylation. Yet another means of improvement of side chain alkylation selectivity can be to treat a molecular sieve zeolite catalyst with chemical compounds capable of providing redox reactions. In one embodiment the catalyst used in an embodiment of the present invention is a basic or neutral catalyst.

The catalytic reaction systems suitable for this invention can include one or more of the zeolite or amorphous materials modified for side chain alkylation selectivity. In an embodiment, the zeolite can be promoted with a first promoter being Cs, wherein the first promoter is capable of facilitating the alkylation reaction, and a second promoter of one or more of Fe, Cu, Ag, V, Cr, Mn, Mo, Sn, Ce, Nb, Bi, Ga, or combinations thereof, capable of facilitating the redox reactions. In another embodiment, the zeolite can be promoted with a first promoter being Cs, wherein the first promoter is capable of facilitating the alkylation reaction, and a second promoter of one selected from the group of Fe, Cu, or combinations thereof, capable of facilitating the redox reaction. Optionally, the zeolite can be promoted with a third promoter of one or more selected from the group of Co, Mn, Ti, Zr, V, Nb, K, Ga, B, P, Rb, Na, Mg, and any combinations thereof. The first, second, or third promoter can exchange with an element within the zeolite or amorphous material and/or be attached to the zeolite or amorphous material in an occluded manner. In an aspect the amount of the first, second, or third promoter is determined by the amount needed to yield less than 0.5 mol % of ring alkylated products such as xylenes from a coupling reaction of toluene and a C₁ source.

In an embodiment, the catalyst contains greater than 0.1 wt % of a plurality of promoters based on the total weight of the catalyst. In another embodiment, the catalyst contains up to 5 wt % of a plurality of promoters. In a further embodiment, the catalyst contains from 0.1 to 3 wt % of a plurality of promoters. In an aspect, the first promoter is cesium.

A zeolite is generally a porous, crystalline alumino-silicate, and it can be formed either naturally or synthetically. For the present invention, the catalyst can be a zeolite, but can also be a non-zeolite. Zeolite materials suitable for this invention may include silicate-based zeolites and amorphous compounds such as faujasites, mordenites, etc. Silicate-based zeolites are made of alternating SiO₂ and MOₓ tetrahedra, where M is an element selected from the Groups 1 through 16 of the Periodic Table (new IUPAC). These types of zeolites have 4, 6, 8, 10, or 12-membered oxygen ring channels.

One method of forming a synthetic zeolite is the hydrothermal digestion of silica, alumina, sodium or other alkyl metal oxide, and an organic templating agent. The amounts of each reactant and the inclusion of various metal oxides can lead to several different synthetic zeolite compositions. Furthermore, a zeolite is commonly altered through a variety of methods to adjust characteristics such as pore size, structure, activity, acidity, and silica/alumina molar ratio. Thus, a number of different forms of zeolite are available. Silicate-based zeolites are made of alternating SiO₂ and MOₓ tetrahedra, where M is an element selected from the Groups 1 through 16 of the Periodic Table (new IUPAC). These types of zeolites have 4-, 6-, 8-, 10-, or 12-membered oxygen ring channels. Other suitable zeolite and zeolite-like catalysts include the types zeolite A, zeolite X, zeolite Y, zeolite L, zeolite beta, zeolite omega, ZSM-5, MCM-22, MCM-41, as well as faujasite, mordenite, chabazite, offretite, clinoptilolite, erionite, sihealite, and the like.

Additionally, it is possible to generate crystals that are not alumino-silicates but behave similarly to zeolite, including aluminophosphates (ALPO) and silicoaluminophosphates (SAPO).

A variety of zeolites and non-zeolites are available for use in the present invention. The various catalysts listed in this disclosure are not meant to be an exhaustive list, but is meant to indicate the type of catalysts for which may be useful in the present invention.

In an embodiment, the zeolite materials suitable for this invention are characterized by silica to alumina ratio (Si/Al) of less than 1.5. In another embodiment, the zeolite materials are characterized by a Si/Al ratio ranging from 1.0 to 200, optionally from 1.0 to 100, optionally from 1.0 to 50, optionally from 1.0 to 10, optionally from 1.0 to 2.0, optionally from 1.0 to 1.5.

The present catalyst is adaptable to use in the various physical forms in which catalysts are commonly used. The catalyst of the invention may be used as a particulate material in a contact bed or as a coating material on structures having a high surface area. If desired, the catalyst can be deposited with various catalyst binder and/or support materials.

A catalyst comprising a substrate that supports a combination of metals can be used to catalyze the reaction of hydrocarbons. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%. The promoters, as combined, generally can range from 0.01% up to 70% by weight of the catalyst. The elements of the catalyst composition can be provided from any suitable source, such as in its elemental form, as a salt, as a coordination compound, etc.

The addition of a support material to improve the catalyst physical properties is possible within the present invention. The powder form of a zeolite and other catalysts may be unsuitable for use in a reactor, due to a lack of mechanical stability, making alkylation and other desired reactions difficult. To render a catalyst suitable for the reactor, it can be combined with a binder to form an aggregate, such as a zeolite aggregate. The binder-modified zeolite, such as a zeolite aggregate, will have enhanced mechanical stability and strength over a zeolite that is not combined with a binder, or otherwise in powder form. The aggregate can then be shaped or extruded into a form suitable for the reaction bed. The binder can desirably withstand temperature and mechanical stress and ideally does not interfere with the reactants adsorbing to the catalyst. In fact, it is possible for the binder to form macropores, much greater in size than the pores of the catalyst, which provide improved diffusional access of the reactants to the catalyst.

Binder material, extrusion aids or other additives can be added to the catalyst composition or the final catalyst composition can be added to a structured material that provides a support structure. For example, the final catalyst composition can include an alumina or aluminate framework as a support. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

Binder materials that are suitable for the present invention include, but are not limited to, silica, alumina, titania, zirconia, zinc oxide, magnesia, boria, silica-alumina, silica-magnesia, chromia-alumina, alumina-boria, silica-zirconia, silica gel, clays, kaolin, montmorillonite, modified clays, similar species, and any combinations thereof. The most frequently used binders are amorphous silica and alumina, including gamma-, eta-, and theta-alumina. It should be noted that a binder can be used with many different catalysts, including various forms of zeolite and non-zeolite catalysts that require mechanical support.

In one embodiment, the catalyst can be prepared by combining a substrate with a plurality of promoter elements. Embodiments of a substrate can be a molecular sieve, from either natural or synthetic sources. Zeolites and zeolite-like materials can be an effective substrate. Alternate molecular sieves also contemplated are zeolite-like materials such as the crystalline silicoaluminophosphates (SAPO) and the aluminophosphates (ALPO).

The present invention is not limited by the method of catalyst preparation, and all suitable methods should be considered to fall within the scope herein. Particularly effective techniques are those utilized for the preparation of solid catalysts. Conventional methods include co-precipitation from an aqueous, an organic or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos. 8/9, pp. 1257-1306, 1995, incorporated herein in its entirety.

The promoter elements can be added to or incorporated into the substrate in any appropriate form. In an embodiment, the promoter elements are added to the substrate by mechanical mixing, by impregnation in the form of solutions or suspensions in an appropriate liquid, or by ion exchange. In a more specific embodiment, the promoter elements are added to the substrate by impregnation in the form of solutions or suspensions in a liquid selected from the group of acetone, anhydrous (or dry) acetone, methanol, and aqueous solutions.

In another more specific embodiment, at least one promoter is added to the substrate by ion exchange. Ion exchange may be performed by conventional ion exchange methods in which sodium, hydrogen, or other inorganic cations that may be typically present in a substrate are at least partially replaced via a fluid solution. In an embodiment, the fluid solution can include any medium that will solubilize the cation without adversely affecting the substrate. In an embodiment, the ion exchange is performed by heating a solution containing a first promoter of Cs to facilitate the alkylation reaction and a second promoter selected from the group of Mn, Fe, Cr, Mo, Sn, Nb, Bi, Ce, V, Ga, Ag, Cu, and any combinations thereof in which the promoter(s) is(are) solubilized in the solution, which may be heated, and contacting the solution with the substrate. The second promoter facilitates the redox reaction. In another embodiment, the ion exchange includes heating a solution containing Cs to facilitate the alkylation reaction and a second promoter selected from the group of Fe and Cu, to facilitate the redox reaction. Optionally, the ion exchange is performed by heating a solution containing a first promoter of Cs to facilitate the alkylation reaction; a second promoter selected from the group of Mn, Fe, Cr, Mo, Sn, Nb, Bi, Ce, V, Ga, Ag, Cu, and any combinations thereof; and a third promoter selected from the group of Co, Mn, Ti, Zr, V, Nb, K, Ga, B, P, Rb, Na, Mg, and any combinations thereof. In an embodiment, the solution is heated to temperatures ranging from 50 to 120° C. In another embodiment, the solution is heated to temperatures ranging from 70 to 100° C.

The solution for use in the ion exchange method may include any fluid medium. A non-fluid ion exchange is also possible and within the scope of the present invention. In an embodiment, the solution for use in the ion exchange method includes an aqueous medium or an organic medium. In a more specific embodiment, the solution for use in the ion exchange method includes water.

The promoters may be incorporated into the substrate in any order or arrangement. In an embodiment, all of the promoters are simultaneously incorporated into the substrate. In more specific embodiment, each promoter is in an aqueous solution for ion-exchange with and/or impregnation to the substrate. In another embodiment, each promoter is in a separate aqueous solution, wherein each solution is simultaneously contacted with the substrate for ion-exchange with and/or impregnation to the substrate. In a further embodiment, each promoter is in a separate aqueous solution, wherein each solution is separately contacted with the substrate for ion-exchange with and/or impregnation to the substrate.

In an aspect, the plurality of promoters includes cesium. In an embodiment, the catalyst contains greater than 0.1 wt % cesium based on the total weight of the catalyst. In another embodiment, the catalyst contains from 0.1 to 33 wt % cesium.

The cesium promoter can be added to the catalyst by contacting the substrate, impregnation, or any other method, with any known cesium source. In an embodiment, the cesium source is selected from the group of nitrates, acetates, oxalates, halides, carbonates, and hydroxides and combinations thereof.

When slurries, precipitates or the like are prepared, they may be dried, usually at a temperature sufficient to volatilize the water or other carrier, such as from 100° C. to 250° C., with or without vacuum. Irrespective of how the components are combined and irrespective of the source of the components, the dried composition is generally calcined in the presence of an oxygen-containing gas, usually at temperatures between about 300° C. and about 900° C. for from 1 to 24 hours. The calcination can be in an oxygen-containing atmosphere, or alternately in a reducing or inert atmosphere.

The prepared catalyst can be ground, pressed, sieved, shaped and/or otherwise processed into a form suitable for loading into a reactor. The reactor can be any type known in the art to make catalyst particles, such as a fixed bed, fluidized bed, or swing bed reactor. Optionally an inert material can be used to support the catalyst bed and to place the catalyst within the bed. Depending on the catalyst, a pretreatment of the catalyst may, or may not, be necessary. For the pretreatment, the reactor can be heated to elevated temperatures, such as 200° C. to 900° C. with an air flow, such as 100 mL/min, and held at these conditions for a length of time, such as 1 to 3 hours. Then, the reactor can be brought to the operating temperature of the reactor, for example 300° C. to 550° C., or optionally down to any desired temperature, for instance down to ambient temperature to remain under a purge until it is ready to be put in service. The reactor can be kept under an inert purge, such as under a nitrogen or helium purge.

Embodiments of reactors that can be used with the present invention can include, by non-limiting examples: fixed bed reactors; fluid bed reactors; and entrained bed reactors. Reactors capable of the elevated temperature and pressure as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to be limiting on the scope of the present invention. An example of a suitable reactor can be a fluid bed reactor having catalyst regeneration capabilities. This type of reactor system employing a riser can be modified as needed, for example by insulating or heating the riser if thermal input is needed, or by jacketing the riser with cooling water if thermal dissipation is required. These designs can also be used to replace catalyst while the process is in operation, by withdrawing catalyst from the regeneration vessel from an exit line or adding new catalyst into the system while in operation.

Upon deactivation, the zeolite may require a regeneration procedure to be performed. Some methods of regenerating a zeolite include heating to remove adsorbed materials, ion exchanging with sodium to remove unwanted cations, or a pressure swing to remove adsorbed gases. One solution involves flushing the catalyst with benzene. Other solutions generally involve processing the catalyst at high temperatures using regeneration gas and oxygen. According to one procedure, a zeolite beta can be regenerated by heating the catalyst first to a temperature in excess of 300° C. in an oxygen-free environment. Then an oxidative regeneration gas can be supplied to the catalyst bed with oxidation of a portion of a relatively porous coke component to produce an exotherm moving through the catalyst bed. Either the temperature or the oxygen content of the gas can be progressively increased to oxidize a porous component of the coke. Again, regeneration gas can be supplied, wherein the gas has either increased oxygen content or increased temperature to oxidize a less porous refractory component of the coke. The regeneration process can be completed by passing an inert gas through the catalyst bed at a reduced temperature.

In another aspect, the reactor may include one or more catalyst beds. In the event of multiple beds, an inert material layer can separate each bed. The inert material can comprise any type of inert substance. In an embodiment, a reactor includes between 1 and 25 catalyst beds. In a further embodiment, a reactor includes between 2 and 10 catalyst beds. In a further embodiment, a reactor includes between 2 and 5 catalyst beds. In addition, the C₁ source and toluene may be injected into a catalyst bed, an inert material layer, or both. In a further embodiment, at least a portion of the C₁ source is injected into a catalyst bed(s) and at least a portion of the toluene feed is injected into an inert material layer(s).

In an alternate embodiment, the entire C₁ source is injected into a catalyst bed(s) and all of the toluene feed is injected into an inert material layer(s). In another aspect, at least a portion of the toluene feed is injected into a catalyst bed(s) and at least a portion the C₁ source is injected into an inert material layer(s). In a further aspect, all of the toluene feed is injected into a catalyst bed(s) and the entire C₁ source is injected into an inert material layer(s).

The feed stream containing the reactants can be treated for the removal of contaminants prior to its introduction into the reaction bed. Various forms of feed stream pretreatment can remove contaminants such as olefins, diolefins, styrene, oxygenated organic compounds, sulfur-containing compounds, nitrogen-containing compounds, and oligomeric compounds. One method is the use of a large pore molecular sieve catalyst to remove impurities prior to the alkylation reaction. The molecular sieve can be placed, for example, in a reactive guard bed, in which the alkylation reaction occurs along with the capture of contaminants. This reactive guard bed can be equipped with a by-pass, so that the molecular sieve can undergo regeneration without interrupting the reaction in the main alkylation reactor beds.

Inert diluents such as helium and nitrogen may be included in the feed to adjust the gas partial pressures. Optionally, CO₂ or water (steam) can be included in the feed stream as these components may have beneficial properties, such as in the prevention of coke deposits. The reaction pressure is not a limiting factor regarding the present invention and any suitable condition is considered to be within the scope of the invention.

The reactants toluene and methanol (and/or formaldehyde) can enter the reactor via a single inlet or separate inlets. The reactants can be delivered to the reaction bed in the gaseous phase, the liquid phase, a combination of liquid and gaseous phase, the supercritical phase, or a combination of liquid and supercritical phases. The reaction conditions, including reactor type, pressure, temperature, liquid hourly space velocity (LHSV), and toluene to methanol ratio depend in part on the phase in which the alkylation is to occur.

Any suitable space velocity, within the short reaction time parameters of the present invention, can be considered to be within the scope of the invention. As used herein the space velocity shall be defined as: space velocity=[feed flow as vapor (cm³/h)]/[catalyst weight (g)]. A standard reference temperature and pressure (72° F. and 14.7 psia) is used to convert a liquid under these conditions, such as toluene, to a feed vapor flow. For example: 0.076 cm³/min of liquid toluene is converted into moles and then using 22.4 L/mol (as if it were an ideal gas) it is converted into a vapor flow of 16 cm³/min. The space velocity can generally range from 5,000 cm³g⁻¹h⁻¹ to 100,000 cm³g⁻¹h⁻¹, optionally from 20,000 cm³g⁻¹h⁻¹ to 85,000 cm³g⁻¹h⁻¹. This range is an indication of possible space velocities, such as for a fixed bed reactor. Of course altering the catalyst composition, the amount of inert material, etc can alter the space velocity outside of this range. Also a change in the reactor from a fixed bed to an alternate design, such as a fluidized bed can also dramatically change the relative space velocity and can be outside of the stated range above. The space velocity ranges given are not limiting on the present invention and any suitable condition is considered to be within the scope of the invention.

The toluene and C₁ source coupling reaction may have a toluene conversion percent greater than 0.01 mol %. In an embodiment the toluene and C₁ source coupling reaction is capable of having a toluene conversion percent in the range of from 0.05 mol % to 40 mol %. In a further embodiment the toluene and C₁ source coupling reaction is capable of having a toluene conversion in the range of from 2 mol % to 40 mol %, optionally from 5 mol % to 35 mol %, optionally from 10 mol % to 30 mol %.

In an aspect the toluene and C₁ source coupling reaction is capable of selectivity to styrene greater than 1 mol %. In another aspect, the toluene and C₁ source coupling reaction is capable of selectivity to styrene in the range of from 1 mol % to 99 mol %. In an aspect the toluene to a C₁ source coupling reaction is capable of selectivity to ethylbenzene greater than 1 mol %. In another aspect, the toluene and C₁ source coupling reaction is capable of selectivity to ethylbenzene in the range of from 1 mol % to 99 mol %. In an aspect the toluene and C₁ source coupling reaction is capable of yielding less than 0.5 mol % of ring alkylated products such as xylenes.

### EXAMPLES

### Example 1

Procedure used to produce the cesium ion-exchanged X-zeolite material: A glass cylinder (2″ inside diameter), fitted with a sintered glass disk and stopcock at the lower end, was charged with 544-HP zeolite (100 g, W.R. Grace) and CsOH (400 mL, 1.0 M in water). The mixture was then brought to 90° C. and allowed to stand for 4 h. The liquid was drained from the zeolite material and another aliquot of CsOH (400 mL of 1.0 M solution in water) was added, heated, and allowed to stand for 3 hours at 90° C. The liquid was drained from the zeolite material and another aliquot of CsOH (400 mL of 1.0 M solution in water) was added, heated, and allowed to stand for 15 hours at 90° C. The liquid was drained from the zeolite material and dried at 150° C. for 1.5 hours.

Incipient wetness impregnation of Ga(NO₃)₃ on to the cesium ion-exchanged X-zeolite material: The cesium ion-exchanged zeolite material (50 g) was subjected to incipient wetness impregnation of Ga(NO₃)₃ by adding the Ga(NO₃)₃ solution (1.83 g Ga(NO₃)₃ in 13.3 mL of water) to the zeolite while stirring. The (Cs, Ga)/X material was then dried at 150° C. for 12 hours.

Stainless steel reactor details: A stainless steel tube with 0.5-inch outer diameter and 0.465 inch internal diameter was filled with crushed quartz of 850-2000 um size (to a height of about 10 inches, 29.2 mL), then the catalyst (to a height of 3.0 inches; 6.6 mL, 3.35 g) at sizes ranging from 250 to 425 μm, and then more crushed quartz of 850 to 2000 μm size (to a height of about 17 inches, 37.2 mL) such that a 0.125 inch stainless steel thermowell was positioned in the middle of the catalyst bed.

Ceramic lined stainless steel reactor details: Experiments were carried out with methanol and toluene over the respective catalyst. A 0.75-inch outside diameter stainless steel tube was fitted with a 0.5-inch inside diameter ceramic liner. The tube was then filled with crushed quartz (to a height of about 13.5 inches), then the catalyst (see Table 1) at sizes ranging from 250 to 425 μm, and then more crushed quartz (of a height of about 17 inches) such that a silcosteel coated thermowell was positioned in the middle of the bed. The reactor was installed in a 3-zone furnace and heated to 500° C. and held for 2 hours while passing nitrogen through it at 150 cc/min. The reactor was then cooled to the reaction temperature of 420° C. The feed was comprised of toluene, methanol and nitrogen. The flow rates were corrected for temperature, the flow rate of gases at the reaction temperature is found in Table 1 as well as the contact time. The effluent was monitored by an on-line gas chromatograph.

The information in Table 1 describes the conditions used in testing various catalysts for producing styrene and ethylbenzene from toluene and methanol:

TABLE 1

| | | | | N₂ | | | | | |
|---|---|---|---|---|---|---|---|---|---|
| | | MeOH | PhMe | (carrier | Tol/MeOH | | | Contact | Time on |
| | Catalyst | (Liq) | (Liq) | gas) | (molar | Temp | Press | Time | stream |
| Catalyst | Size | (mL/hr) | (mL/hr) | (cc/min) | ratio) | (° C.) | psig | (s) | min |
| Cs/X | 250-425 | 4.9 | 13.0 | 20 | 1.0 | 420 | 3.7 | 1.5 | 131 |
| | micron | | | | | | | | |
| Cs/X | 2 mm | 2.3 | 23.0 | 20 | 3.7 | 420 | 5 | 4.1 | 123 |
| Cs, Ga/X | 250-425 | 1.5 | 17.0 | 28 | 4.3 | 420 | 1.8 | 1.6 | 117 |
| | micron | | | | | | | | |
| Cs, Ga/X | 250-425 | 4.9 | 13.0 | 28 | 1.0 | 420 | 2.6 | 1.4 | 318 |
| | micron | | | | | | | | |

Table 2 shows the results of the experiments from Example #1 showing the toluene conversion X_{Tol} and selectivities to ethylbenzene S_{EB}, styrene S_{Sty}, benzene S_{Bz}, and xylenes S_{Xyl}. The X-zeolite based catalyst demonstrated a higher toluene conversion and high EB selectivity over the comparable other zeolite based catalysts. The (Cs, Ga)/X catalyst demonstrated a higher toluene conversion than the Cs/X catalyst.

TABLE 2

| | X_{Tol} | S_{EB} | S_{Sty} | | S_{Bz} | S_{Xyl} |
|---|---|---|---|---|---|---|
| Catalyst | wt % | mol % | mol % | S_{Sty}/S_{EB} | mol % | mol % |
| Cs/X | 7.2 | 83.6 | 8.2 | 0.1 | 0.25 | 0.0 |
| Cs/X | 7.5 | 82.2 | 8.7 | 0.1 | 1.4 | 0.5 |
| Cs, Ga/X | 3.8 | 90.9 | 2.3 | 0.0 | 1.0 | 0.0 |
| Cs, Ga/X | 14.6 | 89.1 | 4.4 | 0.0 | 0.4 | 0.0 |

### Example 2

The following catalysts were prepared: Ref: Cs/X zeolite, Post Cu: Cu—Cs/X, Post Fe: Fe—Cs/X, Pre Cu: Cs—Cu/X, and Pre Fe: Cs—Fe/X.

REF: Cs/X zeolite catalyst preparation: An X-type zeolite (1000 g, Arkema G5-XP) was placed in a large 3-neck round bottom flask and a buffered CsOH solution (3 L of de-ionized water with 503 g of CsOH monohydrate and 144 g of glacial acetic acid) was added to the zeolite. A reflux condenser, thermocouple and mechanical stirrer were placed in the flask necks. The mixture was brought to 90° C. and stirred for 16 hours. The mixture was filtered with a Buchner funnel and the wet cake was returned to the flask and fresh solution was added. The mixture was stirred for 2 hours and then filtered again and returned to the flask. Fresh solution was added and the mixture was stirred for 2 hours. Finally, the wet cake was filtered, dried at 150° C., and calcined at 500° C. for 2 hours.

Post Cu: Cu—Cs/X catalyst preparation: A solution was prepared by buffering 44 mL of de-ionized water with ammonium hydroxide and acetic acid until the pH was at 5. Cu(NO₃)₂-3H₂O (1.45 g) was dissolved in the buffered water. This solution was added to a sample of dry reference catalyst, REF, (65 g) by the incipient wetness method and further dried at 150° C., and then calcined at 400° C. for 2 hours.

Post Fe: Fe—Cs/X catalyst preparation: A solution was prepared by buffering 44 mL of de-ionized water with ammonium hydroxide and acetic acid until the pH was at 5. Fe(NO₃)₃-9H₂O (2.4 g) was dissolved in the buffered water and this solution was added to a sample of dry reference catalyst, REF, (65 g) by the incipient wetness method. This solution was dried at 150° C., and then calcined at 400° C. for 2 hours.

Pre Cu: Cs—Cu/X catalyst preparation: Na/X zeolite (Arkema G5-XP, 260 g) was added to a round-bottom flask. 5.6 g of Cu(NO₃)₂-3H₂O was dissolved in 750 mL of de-ionized water and then added to the zeolite. The round-bottom flask was kept stirring while in a heating mantle set at 90° C. for 2 hours. The slurry was filtered and the powder was returned to the flask. 750 mL of 1M buffered (with 0.8M Acetic Acid) CsOH solution was added to the flask and the exchange was kept at 90° C. for 2 hours for the first Cs exchange step. The slurry was filtered and the powder was returned to the flask. 750 mL of 1M buffered (with 0.8M Acetic Acid) CsOH solution was added to the flask and the exchange was kept at 90° C. for 2 hours for the second Cs exchange step. The slurry was filtered and the powder was returned to the flask. 750 mL of 1M buffered (with 0.8M Acetic Acid) CsOH solution was added to the flask and the exchange was kept at 90° C. for 2 hours for the third Cs exchange step. The slurry was filtered and the wet cake was placed in a large beaker and stirred in 400 mL of 0.01M CsOH solution for 30 minutes at room temperature using a mechanical stirrer. The combination was filtered and dried at ambient temperature for 2-3 hours, then dried overnight in a drying oven at 150° C. The material was calcined by ramping at 25° C./min to 350° C. and holding for 2 hours. The material was then ramped at 25° C./min to 400° C. and held for 4 hours. The material was cooled and the catalyst was bottled.

Pre Fe: Cs—Fe/X catalyst material: Na/X zeolite (Arkema G5-XP, 260 g) was added to a round-bottom flask. 9.3 g of Fe(NO₃)₃-9H₂O was dissolved in 750 mL of de-ionized water and the combination was then added to the zeolite. The round-bottom flask was kept stirring while in a heating mantle set at 90° C. for 2 hours. The slurry was filtered and the powder was returned to the flask. 750 mL of 1M buffered (with 0.8M Acetic Acid) CsOH solution was added to the flask and the exchange was kept at 90° C. for 2 hours for the first Cs exchange step. The slurry was filtered and the powder was returned to the flask. 750 mL of 1M buffered (with 0.8M Acetic Acid) CsOH solution was added to the flask and the exchange was kept at 90° C. for 2 hours for the second Cs exchange step. The slurry was filtered and the powder was returned to the flask. 750 mL of 1M buffered (with 0.8M Acetic Acid) CsOH solution was added to the flask and the exchange was kept at 90° C. for 2 hours for the third Cs exchange step. The slurry was filtered and the wet cake was placed in a large beaker and stirred in 400 mL of 0.01M CsOH solution for 30 minutes at room temperature using a mechanical stirrer. The combination was filtered and then dried at ambient temperature for 2-3 hours, then dried overnight in a drying oven at 150° C. The material was calcined by ramping at 25° C./min to 350° C. and holding for 2 hours. The material was then ramped at 25° C./min to 400° C. and held for 4 hours. The material was cooled and the catalyst was bottled.

For the reactor testing, the catalytic powders were compressed, crushed to 250-425 micron particle size, and tested in a quartz reactor (0.5″ ID). The catalysts (2.9 mL per run) were tested at the reaction conditions of: no pretreatment, 0.8 molar ratio of toluene to methanol, atmospheric pressure, 28 mL/min nitrogen carrier gas, 3 mL/h of total liquid flow (2 mL toluene/h and 1 mL MeOH/h), 420° C. reactor furnace, 125° C. preheater, 1.0 LHSV, a 1.6-1.7 second contact time at reactor conditions, and an L/D=1.2.

The results of the modified samples as shown in Table 3 were as follows:

TABLE 3

| 3 hour | | | | |
|---|---|---|---|---|
| run | % X_{Tol} | % X_{MeOH} | % S_{EB} | % S_{Sty} |
| Ref | 12 | 53 | 88 | 5 |
| post Cu | 15 | 86 | 77 | 18 |
| post Fe | 14 | 55 | 93 | 3 |
| pre Cu | 17 | 58 | 84 | 11 |
| pre Fe | 15 | 53 | 92 | 4 |

All modified samples demonstrated higher toluene activity and total selectivity of ethylbenzene and styrene. The Cu catalysts improved the styrene selectivity.

The term “conversion” refers to the percentage of reactant (e.g. toluene) that undergoes a chemical reaction.

X_{Tol}=conversion of toluene (mol %)=(Tolᵢₙ−Tolₒᵤₜ)/Tolᵢₙ

X_{MeOH}=conversion of methanol (mol %)=(MeOHᵢₙ−MeOHₒᵤₜ)/MeOHᵢₙ

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “selectivity” refers to the relative activity of a catalyst in reference to a particular compound in a mixture. Selectivity is quantified as the proportion of a particular product relative to all other products.

S_{Sty}=selectivity of toluene to styrene (mol %)=Styₒᵤₜ/Tol_{converted}

S_{Bz}=selectivity of toluene to benzene (mol %)=Benzeneₒᵤₜ/Tol_{converted}

S_{EB}=selectivity of toluene to ethylbenzene (mol %)=EBₒᵤₜ/Tol_{converted}

S_{Sty+EB}(MEOH)=selectivity of methanol to styrene+ethylbenzene (mol %)=(Styₒᵤₜ+EBₒᵤₜ)/MeOH_{converted}

The term “zeolite” refers to a molecular sieve containing an aluminosilicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves. Of particular interest are the faujasites. Two types of faujasites are X-zeolite and Y-zeolite.

The various aspects of the present invention can be joined in combination with other aspects of the invention and the listed embodiments herein are not meant to limit the invention. All combinations of various aspects of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

What is claimed is:

1. A method of preparing a catalyst, comprising:
contacting a substrate with at least one solution comprising a first promoter being Cs and at least one solution comprising a second promoter, wherein the contact subjects the substrate to the addition of the first and second promoters, thereby forming the catalyst comprising the first and second promoters, wherein the second promoter is capable of facilitating a redox reaction.

2. The method of claim 1, wherein the substrate is a zeolite.

3. The method of claim 1, wherein the second promoter is selected from the group consisting of Fe, Cr, Ce, Mo, Sn, Bi, Ag, Cu, and combinations thereof.

4. The method of claim 1, wherein the second promoter is selected from the group consisting of Fe, Cu, and combinations thereof.

5. The method of claim 1, further comprising a first solution comprising the first promoter being Cs and a second solution comprising the second promoter, wherein the first solution and second solution simultaneously contact the substrate resulting in simultaneous addition of the first and second promoters to the substrate, resulting in a substrate comprising the first and second promoters.

6. The method of claim 1, further comprising a first solution comprising the first promoter being Cs and a second solution comprising the second promoter, wherein the first solution initially contacts the substrate resulting in the addition of the first promoter resulting in a substrate comprising the first promoter, followed by contacting the substrate comprising the first promoter with the second solution comprising the second promoter, resulting in ion exchange between the cationic sites on the substrate and the second promoter, finally resulting in a substrate comprising the first and second promoters.

7. The method of claim 1, wherein the catalyst comprises the first promoter in amounts ranging from 0.1 wt % to 33 wt % based on the total weight of the catalyst.

8. The method of claim 6, wherein the second promoter is selected from the group consisting of Fe, Cu, and combinations thereof.

9. The method of claim 1, wherein the catalyst is capable of effecting a reaction of at least a portion of a C₁ source with toluene to form a product stream comprising one or more of styrene or ethylbenzene.

10. The method of claim 9, further comprising a first solution comprising the first promoter being Cs and a second solution comprising the second promoter, wherein the first solution initially contacts the substrate resulting in the addition of the first promoter resulting in a substrate comprising the first promoter, followed by contacting the substrate comprising the first promoter with the second solution comprising the second promoter, resulting in ion exchange between the cationic sites on the substrate and the second promoter, finally resulting in a substrate comprising the first and second promoters.

11. The method of claim 10, wherein the second promoter is selected from the group consisting of Fe, Cu, and combinations thereof.

12. The method of claim 10, wherein the substrate further comprises a third promoter selected from the group consisting of Co, Mn, Ti, Zr, V, Nb, K, Ga, B, P, Rb, Na, Mg, and any combinations thereof.

13. A catalyst comprising:
a zeolitic support, and
a plurality of promoters comprising a first promoter being Cs and a second promoter selected from the group consisting of Fe, Cr, Ce, Mo, Sn, Bi, Ag, Cu, and combinations thereof, wherein the second promoter is capable of facilitating a redox reaction.

14. The catalyst of claim 13, wherein the second promoter is selected from the group consisting of Fe, Cu, and combinations thereof.

15. The catalyst of claim 13, wherein the catalyst is capable of effecting a reaction of at least a portion of a C₁ source with toluene to form a product stream comprising one or more of styrene or ethylbenzene, wherein the catalyst is capable of effecting selectivity to styrene of greater than 30 mol %.

16. The catalyst of claim 13, wherein the cesium is present in the catalyst in amounts of from 0.1 to 33 wt % based on the total weight of the catalyst.

17. The catalyst of claim 13, wherein the plurality of promoters are added onto the zeolitic support by ion exchange and wherein the ion exchange is performed in an aqueous medium utilizing water soluble promoter precursors.

18. A process for making styrene comprising:
reacting toluene with the C₁ source in the presence of the catalyst in a reactor to form a product stream comprising ethylbenzene and styrene;
wherein the catalyst comprises a first promoter being Cs and a second promoter supported on a zeolite;
wherein the second promoter is capable of facilitating a redox reaction;
wherein the C₁ source is selected from the group consisting of methanol, formaldehyde, formalin, trioxane, methylformcel, paraformaldehyde, methylal, dimethyl ether, and combinations thereof.

19. The process of claim 18, wherein the second promoter is selected from the group consisting of Fe, Cr, Ce, Mo, Sn, Bi, Ag, Cu, and combinations thereof

20. The process of claim 18, wherein the catalyst further comprises a third promoter selected from the group consisting of Co, Mn, Ti, Zr, V, Nb, K, Ga, B, P, Rb, Na, Mg, and combinations thereof.
